(19) United States
(12) Patent Application Publication
(10) Pub. No.: US 2010/0210833 A1
(43) Pub. Date: Aug. 19, 2010

(54) METHOD OF PRODUCING NUCLEOSIDES

(76) Inventors: Oliver Jungmann, Donaueschingen (DE); Norbert Kraut, Tengen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/682,325

(22) PCT Filed: Oct. 10, 2008
(86) PCT No.: PCT/EP2008/063582

§ 371 Date: Apr. 9, 2010

(30) Foreign Application Priority Data
Oct. 10, 2007 (EP) 07019825.4

(51) Int. Cl. C07H 19/067

(52) U.S. Cl. 536/27.11

(57) ABSTRACT

Method of producing a free nucleoside compound, the compound 2′-deoxy-5-azacytidine (Decitabine) being excluded, by reacting a glycoside donor preferably a 1-halogen derivative, or 1-O-acyl, 1-O-alkyl, or an imidate preferably a trichloromethyl derivative, or a thio-alkyl derivative of a blocked monosaccharide or oligosaccharide preferably ribose and 2-desoxyribose derivatives with a protected nucleoside base, in a suitable anhydrous solvent and in the presence of a catalyst, and removing the protecting groups from said blocked nucleoside compound, wherein said catalyst is selected from the group comprising salts of an aliphatic sulphonic acid and/or salts a strong inorganic acid containing a non-nucleophilic anion.

The present invention refers to a method of producing nucleosides, the compound 2′-deoxy-5-azacytidine (Decitabine) being excluded, by reacting a protected, preferably silylated, nucleoside base with a glycoside donor, preferably a 1-halogen derivative, or 1-O-acyl, 1-O-alkyl, or an imidate preferably a trichloromethyl imidate, or a thio-alkyl derivative of a blocked monosaccharide or oligosaccharide, preferably ribose and 2-desoxyribose derivatives, in the presence of a selected catalyst.

### STATE OF THE ART

Nucleosides are known pharmaceutically active compounds and have been described in numerous publications. From U.S. Pat. No. 3,817,980 it is known to synthesize nucleosides by silylating the corresponding nucleoside base and reacting the silylated base with a glycosyl donor preferably a 1-halogen derivative of a blocked monosaccharide or oligosaccharide in the presence of a selected catalyst. The catalysts used are e.g. selected from SnCl₄, TiCl₄, ZnCl₂, BF₃-etherate, AlCl₂ or SbCl₅. The major disadvantage is that these catalysts are prone to hydrolysis giving irritant hydrolysis products like HCl and/or are forming insoluble oxides (TiO₂, SnO₂), which are difficult to remove from the reaction product. These catalysts and difficult to handle, especially on large scale production.

U.S. Pat. No. 4,082,911 refers to the analogous process of reacting a silylated nucleoside base with a protected derivative of a sugar and proposes to use as catalyst a trialkylsilyl ester of a strong organic acid, such as trimethylsilyl-trifluoromethanesulfonate. U.S. Pat. No. 4,209,613 proposes an improvement for the method disclosed in U.S. Pat. No. 4,082,911 by using a single-step process wherein the trialkylsilyl ester of the strong organic acid, such as trimethylsilyl-trifluoromethanesulfonate, is formed in situ from the free acid by reaction of the free acid with the silylating agent, e.g. trialkylchlorosilane, which is present in the appropriate molar amount. Silylating agents such as trialkylchlorosilane, are very reactive and quickly react to form the trialkylsilyl ester of the free acid present in the reaction mixture.

### DESCRIPTION OF THE INVENTION

It has now been found that a glycoside donor preferably a 1-halogen derivative, or 1-O-acyl, 1-O-alkyl, or an imidate, preferably a trichloromethyl imidate [—NH—(O)C—CCl₃], or a thio-alkyl derivative of a blocked monosaccharide or oligosaccharide, preferably ribose and 2-desoxyribose derivatives, can be reacted with a silylated or alkylated nucleoside base in the presence of a selected catalyst, said catalyst being selected from the group comprising a salt of an aliphatic sulphonic acid and/or a salt of a strong inorganic acid containing a non-nucleophilic anion. There is no need to use an ester compound as a catalyst. This very much simplifies the production of nucleosides as described in the present invention. This type of catalyst is stable under aqueous conditions, easy to handle, does not produce irritant hydrolysis products, and can be easily removed.

Furthermore, using the catalyst of the present invention, the selectivity of the reaction for obtaining the desired anomer, i.e. the ratios of the alpha/beta anomers are excellent. Further, according to the present invention a reaction yield that is higher than 95%, and regularly is within the range of 97-99%, calculated to the total amount of anomers present in the final crude reaction mixture, can be obtained.

The present invention is defined in the claims. The present invention refers to a method of producing a free nucleoside compound, with the proviso that the compound 2′-deoxy-5-azacytidine (Decitabine) is excluded, by reacting a glycoside donor, preferably a 1-halogen, or 1-O-acyl, or a 1-O-alkyl, or an imidate, preferably a trichloromethyl imidate derivative, or a thio-alkyl derivative, preferably a thiomethyl derivative, of a blocked monosaccharide or oligosaccharide, preferably a ribose and/or a 2-desoxyribose derivative, said monosaccharide or oligosaccharide being blocked by removable protecting groups, with a protected nucleoside base, in a suitable anhydrous solvent and in the presence of a catalyst, whereby a blocked nucleoside compound is obtained, and removing the protecting groups from said blocked nucleoside compound in order to obtain the free nucleoside compound, characterized in that said catalyst is selected from the group comprising salts of an aliphatic sulphonic acid and/or salts of a strong inorganic acid containing a non-nucleophilic anion.

The present invention refers also to the production of the blocked nucleoside compound, with the exception of the blocked compound of 2′-deoxy-5-azacytidine (Decitabine), as obtained from the reaction of the glycoside donor preferably a 1-halogen derivative, or 1-O-acyl, or 1-O-alkyl, or an imidate, preferably a trichloromethyl imidate derivative, or a thio-alkyl derivative, preferably a thiomethyl derivative, of a blocked monosaccharide or oligosaccharide, preferably a ribose and/or a 2-desoxyribose derivative, with the protected nucleoside base, characterized in that in said reaction the catalyst is selected from the group comprising salts of an aliphatic sulphonic acid and/or or salts of a strong inorganic acid containing a non-nucleophilic anion.

The catalyst used in said reaction as a salt of an aliphatic sulphonic acid preferably is preferably a salt of methylsulphonic acid (mesylate) or of ethylsulphonic acid, or is a salt of a fluorinated aliphatic sulfonic acid, such as a salt of trifluoromethane-sulfonic acid, of pentafluoroethyl-sulfonic acid, or of heptafluoropropyl-sulfonic acid.

The catalyst used in said reaction as a salt of a strong inorganic acid, is a salt composed of an cation as defined further on herein and a non-nucleophilic anion. Said non-nucleophilic anion does not form a complex with said cation in solution. Preferably said salt of a strong inorganic acid is selected from the group comprising: MBPh₄, MB(Me)₄, MPF₆, MBF₄, MClO₄, MBrO₄, MJO₄, M₂SO₄, MNO₃, and M₃PO₄. (M=metal cation; F=fluorine; Cl=chlorine; Br=bromine; B=boron; Ph=phenyl; Me=methyl; P=phosphorous; J=iodine). Preferred are MBPh₄, MB(Me)₄, MPF₆, MBF₄, MClO₄, MBrO₄, MJO₄, most preferred are the salts of perchloric acid (MClO₄) and of tetrafloroboric acid (MBF₄). Most preferred are the salts wherein M is lithium.

Preferred of these salts are the salts of methylsulphonic acid (mesylate), the salts of trifluoromethanesulfonic acid, and the salts of perchloric acid.

Preferred aliphatic sulphonic acid salts, fluorinated aliphatic sulfonic acid salts and salts of a strong inorganic acid are the alkali salts and earth alkali salts, preferably the salts of lithium, sodium, potassium, or magnesium. Preferred are the lithium salts, preferably lithium methylsulphonic acid (lithium mesylate), lithium-trifluoromethanesulfonate (LiOTf, lithium-triflate) and lithium perchlorate (LiClO₄). Also other salts, for example the salts of scandium, such as Sc(OTf)₃ or of copper such as Cu(OTf)₂ or of magnesium such as Mg(OTf)₂ can be used. However, the lithium salt and especially LiOTf is preferred.

Preferred solvents to carry out the reaction according to the present invention are organic solvents such as benzene, toluene, xylol, or chlorinated solvents, for example dichloromethane, dichloroethane, chloroform, chlorobenzene, or acetonitril and/or propylene carbonate and/or related solvents. Preferred are toluene and chlorinated solvents. Preferred is the use of lithium-trifluoromethanesulfonate (LiOTf) in a chlorinated solvent, preferably in dichloromethane, dichloroethane, chloroform, chlorobenzene and/or in an aromatic solvent like toluene or xylene. Each solvent or mixture of solvents may yield a different selectivity with respect to the beta-isomer (β-isomer). It is no problem for the expert in the art to optimize the catalyst and/or solvent or the mixture of solvents in order to obtain the desired selectivity in favor of the beta-isomer.

The synthesis of glycoside donors as defined herein is known per se. The glycoside donor preferably is a 1-halogen derivative, or 1-O-acyl, or a 1-O-alkyl, or an imidate preferably a trichloromethyl imidate derivative, or a thio-alkyl derivative of a blocked monosaccharide or oligosaccharide, preferably of a blocked ribose and/or 2-desoxyribose, wherein the hydroxyl groups are being blocked by protecting groups, i.e. removable substituents. Such compounds and numerous substituents are known and can be used within the scope of the present invention.

Said protecting groups preferably are selected from (C₁-C₈)alkylcarbonyl, or optionally substituted phenylcarbonyl, or optionally substituted benzylcarbonyl. Preferably, said protecting groups are selected from (C₁-C₄)alkylcarbonyl, or optionally substituted phenylcarbonyl, like phenylcarbonyl, tolylcarbonyl, xylylcarbonyl or benzylcarbonyl; and is preferably acetyl or p-chloro-phenylcarbonyl.

The substituents 1-O-acyl, 1-O-alkyl, 1-halogen, 1-imidate or 1-thio-alkyl attached to the blocked monosaccharide or oligosaccharide are preferably substituents of the formulae —O-acyl(C₁-C₈), —O-alkyl(C₁-C₈) or halogen or trichloromethyl imidate, or thiomethyl; preferably are —O-acyl(C₁-C₄), —O-alkyl(C₁-C₄) or chlorine, bromine, fluorine; preferably —O—(O)C—CH₃ or chlorine, bromine, fluorine, preferably chlorine or fluorine, preferably chlorine.

The blocked monosaccharide or oligosaccharide are preferably derived from ribose, deoxyribose, arabinose, and glucose, preferably from ribose and 2-desoxyribose. Preferably all free hydroxyl groups are blocked with known protecting groups, preferably with the blocking groups as mentioned herein above, selected from (C₁-C₈)alkylcarbonyl, or optionally substituted phenylcarbonyl, or optionally substituted benzylcarbonyl. These blocking groups are known to the expert in the art.

The protected nucleoside base is protected by a removable protecting group known per se, and is preferably protected by a trimethylsilyl (TMS)-group. The preparation of protected nucleoside base compounds is known. The compound is preferably prepared by reaction of the free nucleoside base with trimethylchlorosilane or with hexamethyldisilazane. This is known to the expert in the art. Numerous nucleoside organic bases are known. Generally all these nucleoside organic bases can be reacted with the corresponding chemical compounds, e.g. trimethylchlorosilane or with hexamethyldisilazane, to yield the protected nucleoside base which can be used according to the process defined in the present invention.

Preferred nucleoside bases are halogen-derivatives, preferably chlor- or fluor-substituted derivatives, preferably fluorine substituted, and heterocyclic compounds containing five or six atoms, said heterocyclic ring containing one, two or three nitrogen atoms. Preferably the nucleoside bases are or are derived from the group comprising the following heterocyclic compounds, wherein these compounds optionally may be substituted: uracil, cytosine preferably 5-azacytosine, 6-azauracil, 2-thio-6-azauracil, thymine, N-acyl-adenine, guanine, lumazine, imidazole, pyrazine, thiazole and triazole. In the reaction of the nucleoside base with the glycoside donor, the sugar residue is preferably linked to the nitrogen atom to form a beta-glycoside.

When reacting the protected nucleoside base with the above mentioned derivative of a blocked monosaccharide or oligosaccharide together, the reaction temperature generally is within the range of 0° C. to about 90° C., preferably at about room temperature, whereby the components are reacted in about equimolar amounts and preferably with a slight excess of the protected nucleoside base. The catalyst is used preferably in a concentration of about 10 to 100 mol-%, calculated to the total molar presence of the two reacting components. For the expert in the art it is no problem to optimize the molar ratios of the components.

For removing the substituents from the blocked nucleoside compound in order to obtain the free nucleoside compound, containing free hydroxyl groups, known methods are used. The substituents may be preferably removed, for example, by treatment in an alcoholic solution of ammonia or alcoholates, or with aqueous or alcoholic alkali; but other known methods may be applied. The following example illustrates the invention.

### Example 1

(A) A mixture of cytosine (2.5 g, 22.5 mmol), ammonium sulfate (0.30 g, 2.27 mmol), and hexamethyldisilazane (20.0 g, 123.9 mmol) was heated to reflux for 17 h. A clear solution was obtained. Some gas evolved (ammonia). The reaction mixture was cooled to 52° C. and concentrated in the vacuum whereby a colourless solid precipitated. 25 ml of dichloromethane, lithium trifluoromethane sulfonate (3.51 g, 22.5 mmol) and 1-chloro-3,5-di-o-p-chlorobenzoyl-2-deoxy-α-D-ribofuranose (9.67 g, 22.5 mmol) were added. The slightly beige mixture was stirred for 17 hours at ambient temperature (20-25° C.), products (reaction yield combined anomers: 99.1%; selectivity alpha/beta 38.5/61.5)

(B) Then the solvent was removed at 38° C. A brownish solid was obtained. The solid was dissolved in 7.5 g ethyl acetate. The solution was added drop wise to a mixture of 27.5 g of aqueous sodium hydrogen carbonate (2.5 weight % solution in water), 21.8 g of ethyl acetate, 4.5 g of cyclohexane and 8.8 g of acetonitrile at 20° C. The obtained reaction mixture was stirred at ambient temperature for 1 h, and then for 1.5 hours at 0° C. The precipitate of the blocked (protected) nucleoside was filtered off, washed with 10 g of water, and finally with 20 g of a mixture of acetonitrile and ethyl acetate (1:1).

Yield: 9.90 g; 87.2% combined anomers; ratio alpha/beta 41/59; purity 98.83%.

### Example 2

(A) A mixture of 5-fluoro-cytosine (1.0 g, 7.75 mmol), ammonium sulfate (0.12 g, 0.91 mmol), and hexamethyldisilazane (8.0 g, 49.6 mmol) was heated to reflux for 17 hours. A clear solution was obtained. Some gas evolved (ammonia). The reaction mixture was cooled to 50° C., and concentrated in the vacuum to about half of the original volume. A turbid suspension was obtained. 10 ml of dichloromethane, lithium trifluoromethane sulfonate (1.21 g, 7.75 mmol) and 1-chloro-3,5-di-o-p-chloro-benzoyl-2-deoxy-α-D-ribofuranose (3.33 g, 7.75 mmol) were added. The beige mixture was stirred for 17 hours at ambient temperature (20-25° C.) (reaction yield combined anomers: 99.0%; selectivity alpha/beta 30/70]).

(B) Then the solvent was removed at 38° C. A brownish sticky solid was obtained. The solid was dissolved in 10 g ethyl acetate. The solution was added to 10 g of aqueous hydrogen carbonate (2.5 weight % solution in water). The reaction mixture was stirred at ambient temperature for 2.5 hours, and then filtered off, washed with 3.0 g of water, and finally with 3.6 g ethyl acetate

Yield: 1.80 g; 44.5%; alpha/beta 1.2/98.8; purity: 98.5% beta anomer.

### Example 3

(A) A mixture of 5-azacytosine (0.25 g, 2.23 mmol), ammonium sulfate (0.03 g, 0.23 mmol), and hexamethyldisilazane (2.0 g, 12.4 mmol) was heated to reflux for 17 hours. A clear solution was obtained. Some gas evolved (ammonia). The reaction mixture was cooled to 50° C., and concentrated in the vacuum to about half of the original volume. A turbid suspension was obtained. 3.3 g of dichloromethane, lithium trifluoromethane sulfonate (0.35 g, 2.24 mmol) and 2,3,5-tri-O-benzoyl-alpha-D-arabinofuranosyl-bromide (1.17 g, 2.23 mmol) were added. The beige mixture was stirred for 17 hours at ambient temperature (20-25° C.) (reaction yield: 95.2%; selectivity: 0.1/99.9).

(B) Then the solvent was removed at 38° C. A brownish sticky solid was obtained. The solid was dissolved in 7.0 g ethyl acetate and the reaction mixture was washed with 7.0 g of aqueous hydrogen carbonate (2.5 weight % solution in water). The organic phase was filtered, dried over Na₂SO₄ and evaporated under vacuum. The residue was dissolved in 3.0 g ethyl acetate, 4.0 g cyclohexane was added and the mixture was stirred for 2 hours at ambient temperature. The crystallized product was filtered off and washed with a small amount of a 1:1 mixture of ethyl acetate and cyclohexane.

Yield: 0.40 g; 32.2%; purity: 99.2%

### Example 4

(A) A mixture of 5-Azacytosine (0.25 g, 2.23 mmol), ammonium sulfate (0.03 g, 0.23 mmol), and hexamethyldisilazane (2.0 g, 12.4 mmol) was heated to reflux for 17 hours. A clear solution was obtained. Some gas evolved (ammonia). The reaction mixture was cooled to 50° C., and concentrated in the vacuum to about half of the original volume. A turbid suspension was obtained. 3.3 g of dichloromethane, lithium trifluoromethane sulfonate (0.35 g, 2.24 mmol) and 2,3,5-Tri-O-benzoyl-alpha-D-arabinofuranosyl-bromide (1.17 g, 2.23 mmol) were added. The beige mixture was stirred for 17 hours at ambient temperature (20-25° C.) (Reaction Yield: 94.2%).

(B) The solvent was removed at 38° C. To the oily residue 4.0 g of ethyl acetate were added and then the turbid solution was filtered. The filtrate was quenched with 4.0 g aqueous sodium hydrogen carbonate solution (2.5%-weight). Afterwards 4.0 g of cyclohexane were added and the mixture was cooled to 0-5° C. for 4 hours. The solid was filtered off (5-Azacytosine) and washed with a mixture of ethyl acetate and cyclohexane (1:1). The filtrate was put in a separatory funnel and the product containing organic phase was separated and dried over sodium sulfate. Afterwards the solvent was removed under vacuum and diethyl ether was added to the oily residue, whereas a solid was formed. After stirring for 24 hours the precipitation was filtered off and washed with diethyl ether.

Yield: 0.70 g; 56.3%; Purity: 91.5%

### Example 5

Examples 1 to 4 are repeated replacing lithium trifluoromethane sulfonate each time by the same amount (in equivalents) of one of the compounds lithium mesylate, lithium perchlorate, lithium tetrafluroborate, sodium trifluoromethane sulfonate, potassium trifluoromethane sulfonate and zinc trifluoromethane sulfonate, whereby analogous results are obtained as reported in Examples 1-4.

### Example 6

Examples 1 to 4 are repeated replacing dichloromethane as solvent each time by the same volume of one of the following solvents: toluene, xylene, 1,2-dichloroethane, chlorobenzene, 1,2-dichlorobenzene, acetonitrile and propylenecarbonate, whereby analogous results are obtained as reported in Examples 1-4.

1-21. (canceled)

22. A method of producing a free nucleoside compound, with the proviso that the compound 2′-deoxy-5-azacytidine (Decitabine) is excluded, said method comprising reacting a glycoside donor, preferably a 1-halogen derivative, or 1-O-acyl, 1-O-alkyl, or an imidate, preferably a trichloromethyl imidate, or a thioalkyl derivative preferably a thiomethyl derivative of a blocked monosaccharide or oligosaccharide, preferably ribose and 2-desoxyribose derivatives, said monosaccharide or oligosaccharide being blocked by removable protecting groups, with a protected nucleoside base, in a suitable anhydrous solvent and in the presence of a catalyst, whereby a blocked nucleoside compound is obtained, and removing the protecting groups from said blocked nucleoside compound in order to obtain the free nucleoside compound, wherein catalyst is selected from the group comprising salts of an aliphatic sulphonic acid and/or salts of a strong inorganic acid containing a non-nucleophilic anion.

23. A method of producing a blocked nucleoside compound, with the proviso that the blocked compound of 2′-deoxy-5-azacytidine (Decitabine) is excluded, said method comprising reacting a glycoside donor, preferably a 1-halogen derivative, or 1-O-acyl, 1-O-alkyl, or an imidate preferably a trichloromethyl imidate, or a thio-alkyl derivative of a blocked monosaccharide or oligosaccharide, preferably ribose and 2-desoxyribose derivatives, said monosaccharide or oligosaccharide being blocked by removable protecting groups, with a protected nucleoside base, in a suitable anhydrous solvent and in the presence of a catalyst, whereby a blocked nucleoside compound is obtained, wherein said catalyst is selected from the group comprising salts of an aliphatic sulphonic acid and/or or salts of a strong inorganic acid containing a non-nucleophilic anion.

24. The method according to claim 22,wherein the protecting groups of blocked monosaccharides or oligosaccharides are selected from (C₁-C₈)alkylcarbonyl, or optionally substituted phenylcarbonyl, or optionally substituted benzylcarbonyl, preferably from (C₁-C₄)alkylcarbonyl, or optionally substituted phenylcarbonyl, preferably phenylcarbonyl, tolylcarbonyl, xylylcarbonyl or benzylcarbonyl; and preferably is acetyl or p-chloro-phenylcarbonyl.

25. The method according to claim 22, wherein the substituents 1-O-acyl, 1-O-alkyl and 1-halogen attached to the blocked monosaccharide or oligosaccharide are preferably substituents of the formulae —O-acyl(C₁-C₈), —O-alkyl(C₁-C₈) or halogen, preferably chlorine.

26. The method according to claim 25, characterized in that the substituents 1-O-acyl, 1-O-alkyl and 1-halogen are —O-acyl(C₁-C₄), —O-alkyl(C₁-C₄) or chlorine, preferably —O—(O)CCH₃ or chlorine, preferably chlorine.

27. The method according to claim 22, wherein the blocked monosaccharides or oligosaccharides are derived from ribose, 2-deoxyribose, arabinose, and glucose and wherein preferably all free hydroxyl groups are blocked with known protecting groups, preferably with the blocking groups selected from (C₁-C₈)alkylcarbonyl, or optionally substituted phenylcarbonyl, or optionally substituted benzylcarbonyl.

28. The method according to claim 22, wherein the protected nucleoside base is protected by a removable protecting group known per se, and is preferably protected by a trimethylsilyl (TMS)-group.

29. The method according to claim 22, wherein the nucleoside base is a halogen derivative, preferably Fluor derivatives, or is a heterocyclic compound containing five or six atoms, said heterocyclic ring containing one, two or three nitrogen atoms.

30. The method according to claim 22, wherein the nucleoside base is a halogen derivative, preferably Fluor derivatives, or is derived from the group comprising the following heterocyclic compounds, wherein these compound optionally may be substituted: uracil, cytosine preferably 5-azacytosine, 6-azauracil, 2-thio-6-azauracil, thymine, N-acyl-adenine, guanine, lumazine, imidazole, pyrazine, thiazole and triazole.

31. The method according to claim 22, wherein in the nucleoside obtained, the sugar residue is linked to the nitrogen atom to form a beta-glycoside.

32. The method according to claim 23,wherein the protecting groups of blocked monosaccharides or oligosaccharides are selected from (C₁-C₈)alkylcarbonyl, or optionally substituted phenylcarbonyl, or optionally substituted benzylcarbonyl, preferably from (C₁-C₄)alkylcarbonyl, or optionally substituted phenylcarbonyl, preferably phenylcarbonyl, tolylcarbonyl, xylylcarbonyl or benzylcarbonyl; and preferably is acetyl or p-chloro-phenylcarbonyl.

33. The method according to claim 23, wherein the substituents 1-O-acyl, 1-O-alkyl and 1-halogen attached to the blocked monosaccharide or oligosaccharide are preferably substituents of the formulae —O-acyl(C₁-C₈), —O-alkyl(C₁-C₈) or halogen, preferably chlorine.

34. The method according to claim 33, characterized in that the substituents 1-O-acyl, 1-O-alkyl and 1-halogen are —O-acyl(C₁-C₄), —O-alkyl(C₁-C₄) or chlorine, preferably —O—(O)CCH₃ or chlorine, preferably chlorine.

35. The method according to claim 23, wherein the blocked monosaccharides or oligosaccharides are derived from ribose, 2-deoxyribose, arabinose, and glucose and wherein preferably all free hydroxyl groups are blocked with known protecting groups, preferably with the blocking groups selected from (C₁-C₈)alkylcarbonyl, or optionally substituted phenylcarbonyl, or optionally substituted benzylcarbonyl.

36. The method according to claim 23, wherein the protected nucleoside base is protected by a removable protecting group known per se, and is preferably protected by a trimethylsilyl (TMS)-group.

37. The method according to claim 23, wherein the nucleoside base is a halogen derivative, preferably Fluor derivatives, or is a heterocyclic compound containing five or six atoms, said heterocyclic ring containing one, two or three nitrogen atoms.

38. The method according to claim 23, wherein the nucleoside base is a halogen derivative, preferably Fluor derivatives, or is derived from the group comprising the following heterocyclic compounds, wherein these compound optionally may be substituted: uracil, cytosine preferably 5-azacytosine, 6-azauracil, 2-thio-6-azauracil, thymine, N-acyl-adenine, guanine, lumazine, imidazole, pyrazine, thiazole and triazole.

39. The method according to claim 23, wherein in the nucleoside obtained, the sugar residue is linked to the nitrogen atom to form a beta-glycoside.

40. The method according to any one of claims 22-39, wherein the catalyst used in said reaction is a salt of an aliphatic sulphonic acid, or a salt of a fluorinated aliphatic sulfonic acid.

41. The method according to claim 40, wherein the catalyst used in said reaction is a salt of methylsulphonic acid or a salt of ethylsulphonic acid.

42. The method according to claim 40, wherein the catalyst used in said reaction is a salt of trifluoromethane-sulfonic acid, a salt of pentafluoroethyl-sulfonic acid, or a salt of heptafluoropropyl-sulfonic acid.

43. The method according to any one of claims 22-39, wherein the catalyst is an alkali salt or an earth alkali salt.

44. The method of claim 43, wherein the catalyst is a salt of lithium, a salt of sodium, a salt of potassium, or a salt of magnesium.

45. The method of claim 43, wherein the catalyst is lithium methylsulphonic acid and/or lithium-trifluoromethanesulfonate.

46. The method of claim 43, wherein the catalyst is chosen from the salts comprising salts of scandium, of zinc or of copper.

47. The method of claim 43, wherein the catalyst is Sc(OTf)₃, Zn(OTf)₂, or Cu(OTf)₂.

48. The method according to any one of claims 22-39, wherein the catalyst is a salt of a strong inorganic acid composed of an cation and a non-nucleophilic anion which does not form a complex with said cation in solution.

49. The method of claim 48, wherein the catalyst is selected from the group comprising: MBPh₄, MB(Me)₄, MPF₆, MBF₄, MClO₄, MBrO₄, MJO₄, M₂SO₄, MNO₃, and M₃PO₄.

50. The method of claim 48, wherein the catalyst is a salt of perchloric acid and/or a salt of tetrafloroboric acid.

51. The method according to any one of claims 22-39, wherein the solvent to carry out the reaction is chosen from the group comprising organic solvents or chlorinated solvents, xylol, or acetonitril, propylene carbonate.

52. The method of claim 51, wherein the solvent is benzene, toluene or xylene.

53. The method of claim 51, wherein the solvent is dichloromethane, dichloroethane, chloroform, or chlorobenzene.

54. The method according to any one of claims 22-39, wherein the catalyst is lithium-trifluoromethanesulfonate and the solvent is chosen from toluene, xylene, dichloromethane, dichloroethane, chloroform or chlorobenzene.
